(19) United States
(12) Patent Application Publication
(10) Pub. No.: US 2026/0315736 A1
(43) Pub. Date: Oct. 15, 2026

(54) CATHETER

(71) Applicant: KANEKA CORPORATION, Osaka (JP)

(72) Inventor: Tomoya Kato, Osaka (JP)

(73) Assignee: KANEKA CORPORATION, Osaka (JP)

(21) Appl. No.: 19/459,267

(22) Filed: Jan. 26, 2026

Related U.S. Application Data
(63) Continuation of application No. PCT/JP2024/024770, filed on Jul. 9, 2024.

(30) Foreign Application Priority Data
Aug. 1, 2023 (JP) 2023-125557

(51) Int. Cl. A61M 25/00

(52) U.S. Cl. CPC A61M 25/0026; A61M 2025/0004; A61M 2025/0037; A61M 2025/0039

(57) ABSTRACT

A catheter includes a first tubular member, a second tubular member, and an outer member including a portion that encloses at least a portion of the first tubular member and the second tubular member. The distal end of the first tubular member is located on the proximal side of the distal end of the second tubular member, and the proximal end of the first tubular member is located on the proximal side of the proximal end of the second tubular member. The outer member has a first region distal to the proximal end of the second tubular member and a second region located on the distal side of the first region. The ratio of the major axis to the minor axis of the first region is 1.0 to 1.1, and the ratio of the major axis to the minor axis of the second region is 1.2 to 2.0.

### TECHNICAL FIELD

One or more embodiments of the present invention relate to a catheter.

### BACKGROUND

A catheter that can be inserted into a blood vessel or the like is used to examine and/or treat a lesion site of a human body. As such a catheter, for example, PTL 1 discloses a catheter including a first tubular member that extends in a longitudinal direction, has a distal end and a proximal end, and into which a wire is inserted, and a second tubular member that has a distal end and a proximal end and is adjacent to the first tubular member in a radial direction. The distal end of the first tubular member is located on the proximal side of the distal end of the second tubular member. The proximal end of the first tubular member is located on the proximal side of the proximal end of the second tubular member. The first tubular member includes a reinforcing portion including a reinforcing member and an inner layer containing at least one selected from the group consisting of polytetrafluoroethylene and a tetrafluoroethylene-perfluoroalkyl vinyl ether copolymer. The first tubular member includes, on the distal side of the distal end of the reinforcing member, a resin extension portion that does not include the polytetrafluoroethylene, the tetrafluoroethylene-perfluoroalkyl vinyl ether copolymer, and the reinforcing member.

### PATENT LITERATURE

PTL 1: International Publication No. 2020/255737

### SUMMARY

An existing catheter, such as that disclosed in PTL 1, may be difficult to be inserted into a blood vessel such as a coronary artery in which three-dimensional meandering is continuous. One or more embodiments of the present invention have been made in view of the above circumstance and a catheter that can be easily inserted into a meandering portion in the body is provided.

A catheter according to one or more embodiments of the present invention capable of addressing the above is as follows.
[1] A catheter including:
<TB>a first tubular member extending in a longitudinal direction;
a second tubular member extending in the longitudinal direction and adjacent to the first tubular member in a radial direction; and
an outer member extending in the longitudinal direction and including a portion that encloses at least a portion of the first tubular member and at least a portion of the second tubular member,
wherein a distal end of the first tubular member is located on a proximal side of a distal end of the second tubular member,
wherein a proximal end of the first tubular member is located on a proximal side of a proximal end of the second tubular member, and
wherein the outer member has a first region distal to the proximal end of the second tubular member and extending in the longitudinal direction and a second region extending in the longitudinal direction and located on a distal side of the first region, wherein a ratio of a major axis to a minor axis of the first region in a cross-section in the radial direction is 1. 0 to 1. 1, and a ratio of a major axis to a minor axis of the second region in a cross-section in the radial direction is 1.2 to 2.0.

As a result of studies by the present inventor(s), it has been found that a catheter, such as that described in PTL 1, having a circular cross-sectional shape in the radial direction thereof is difficult to be curved, at a meandering portion in which three-dimensional meandering continues as in a coronary artery, along the shape of the meandering portion. Further, as a result of studies by the present inventor(s), it has been found that, when the cross-sectional shape of a catheter in the radial direction is an elliptical shape, the catheter is easily curved along the shape of a meandering portion but is difficult to be pushed. As a result of further studies by the present inventor(s), it has been found that, when a second region in which a value obtained by dividing the major axis by the minor axis thereof in a cross-section in the radial direction is 1.2 to 2.0 is disposed, in a catheter, on the distal side of a first region in which a value obtained by dividing the major axis by the minor axis thereof is 1.0 to 1.1, the catheter is easily curved along the shape of a meandering portion and is easily pushed. Consequently, the catheter is easily inserted into a meandering portion in the body.

Further, the catheter according to an embodiment may be the catheter in any one of [2] to [22] below.
[2] The catheter according to [1],
wherein a shape of an outer edge of the first region in a cross-section in the radial direction is a circle or an ellipse, and
wherein a shape of an outer edge of the second region in a cross-section in the radial direction is an ellipse or a shape of a portion of an ellipse.
[3] The catheter according to [1] or [2], wherein the outer member has a third region between the first region and the second region in the longitudinal direction, and a ratio of a major axis to a minor axis of the third region in a cross-section in the radial direction is greater than 1.1 and less than 1.2.
[4] The catheter according to any one of [1] to [3], wherein a length of the second region in the longitudinal direction is shorter than a length of the first region in the longitudinal direction.
[5] The catheter according to any one of [1] to [4], wherein an area of a region surrounded by an outer edge of the first region in a cross-section in the radial direction is larger than or equal to an area of a region surrounded by an outer edge of the second region in a cross-section in the radial direction.
[6] The catheter according to any one of [1] to [5], wherein the major axis of the second region is greater than or equal to the major axis of the first region.
[7] The catheter according to any one of [1] to [5], wherein the major axis of the second region is shorter than the major axis of the first region.
[8] The catheter according to any one of [1] to [7], wherein, in the radial direction, a shortest distance from an outer edge of the second region of the outer member to an outer edge of the first tubular member is longer than or equal to a shortest distance from an outer edge of the first region of the outer member to the outer edge of the first tubular member.
[9] The catheter according to any one of [1] to [7], wherein, in the radial direction, a shortest distance from an outer edge of the second region of the outer member to an outer edge of the first tubular member is shorter than a shortest distance from an outer edge of the first region of the outer member to the outer edge of the first tubular member.
[10] The catheter according to any one of [1] to [9], wherein, in the radial direction, a shortest distance from an outer edge of the second region of the outer member to an outer edge of the second tubular member is longer than or equal to a shortest distance from an outer edge of the first region of the outer member to the outer edge of the second tubular member.
[11] The catheter according to any one of [1] to [9], wherein, in the radial direction, a shortest distance from an outer edge of the second region of the outer member to an outer edge of the second tubular member is shorter than a shortest distance from an outer edge of the first region of the outer member to the outer edge of the second tubular member.
[12] The catheter according to any one of [1] to [11], wherein the outer member is tubular and has a lumen extending in the longitudinal direction, and at least a portion of the first tubular member and at least a portion of the second tubular member are disposed in the lumen.
[13] The catheter according to any one of [1] to [11],
wherein the outer member includes a columnar body extending in the longitudinal direction and containing a resin, and
wherein at least a portion of the first tubular member and at least a portion of the second tubular member are disposed in the columnar body.
[14] The catheter according to any one of [1] to [13], further including:
an outer tubular member extending in the longitudinal direction,
wherein the outer tubular member has a lumen extending in the longitudinal direction, and a portion of the first tubular member is disposed in the lumen.
[15] The catheter according to [14], wherein the outer tubular member is located on a proximal side of the proximal end of the second tubular member.
[16] The catheter according to [14] or [15], wherein the outer tubular member has a fourth region proximal to the proximal end of the second tubular member and extending in the longitudinal direction, and wherein a ratio of a major axis to a minor axis of the fourth region in a cross-section in the radial direction is 1.0 to 1.1.
[17] The catheter according to [16], wherein the major axis of the second region is shorter than the major axis of the fourth region.
[18] The catheter according to [16] or [17], wherein a length of the fourth region in the longitudinal direction is longer than a sum of a length of the first region in the longitudinal direction and a length of the second region in the longitudinal direction.
[19] The catheter according to any one of [16] to [18], further including a wire member extending in the longitudinal direction and disposed in at least the first region and the fourth region, wherein the wire member has a distal end located in the first region.
[20] The catheter according to any one of [1] to [19], wherein the outer member has a fifth region extending in the longitudinal direction and located on a distal side of the second region, and a ratio of a major axis to a minor axis of the fifth region in a cross-section in the radial direction is 1.0 to 1.1.
[21] The catheter according to any one of [1] to [20], wherein the first tubular member and the second tubular member are fixed to each other in the radial direction.
[22] The catheter according to any one of [1] to [21], wherein a handle portion is disposed at a proximal end portion of the first tubular member.

According to one or more embodiments of the present invention, with the above-described configurations, it is possible to provide a catheter that can be easily inserted into a meandering portion in the body.

### BRIEF DESCRIPTION OF DRAWINGS

FIG. 1 is a side view of a catheter according to a first embodiment.

FIG. 2 is a longitudinal sectional view of the region R1 in FIG. 1.

FIG. 3 is a longitudinal sectional view of the region R2 in FIG. 1.

FIG. 4 is a cross-sectional view of the catheter taken along line A-A in FIG. 1.

FIG. 5 is a cross-sectional view of the catheter taken along line B-B in FIG. 1.

FIG. 6 is a cross-sectional view of the catheter taken along line C-C in FIG. 1.

FIG. 7 is a cross-sectional view of the catheter taken along line D-D in FIG. 1.

FIG. 8 is a cross-sectional view of the catheter taken along line E-E in FIG. 1.

FIG. 9 is a longitudinal sectional view of the region R2 in a modification of the catheter in FIG. 1.

FIG. 10 is a side view of a catheter according to a second embodiment.

FIG. 11 is a longitudinal sectional view of the region R1 in FIG. 10.

FIG. 12 is a longitudinal sectional view of the region R2 in FIG. 10.

FIG. 13 is a cross-sectional view of the catheter taken along line A-A in FIG. 10.

FIG. 14 is a cross-sectional view of the catheter taken along line B-B in FIG. 10.

FIG. 15 is a cross-sectional view of the catheter taken along line C-C in FIG. 10.

FIG. 16 is a cross-sectional view of a modification of the catheter in FIG. 5.

### DETAILED DESCRIPTION

Hereinafter, one or more embodiments of the present invention will be described more specifically on the basis of the following embodiments. However, needless to say, the present invention is not limited by the following embodiments and can be naturally carried out with appropriate changes within the scope that can be adapted to the gist described above and below, and all of them are included in the technical scope of the present invention. In each of the drawings, reference signs and the like of members may be omitted for convenience. In such a case, the specification and other drawings are referred to. In addition, the dimensions of various members in the drawings may be different from actual dimensions thereof for facilitating understanding of the features of one or more embodiments of the present invention.

A catheter according to an embodiment is a catheter including a first tubular member extending in a longitudinal direction; a second tubular member extending in the longitudinal direction to be adjacent to the first tubular member in a radial direction; an outer member extending in the longitudinal direction and including a portion that encloses at least a portion of the first tubular member and at least a portion of the second tubular member. The distal end of the first tubular member is located on the proximal side of the distal end of the second tubular member, and the proximal end of the first tubular member is located on the proximal side of the proximal end of the second tubular member. The outer member has a first region distal to the proximal end of the second tubular member and extending in the longitudinal direction and a second region extending in the longitudinal direction and located on the distal side of the first region. The value of the ratio of the major axis to the minor axis of the first region in a cross-section in the radial direction is 1.0 to 1.1, and the value of the ratio of the major axis to the minor axis of the second region in a cross-section in the radial direction is 1.2 to 2.0.

As a result of studies by the present inventor(s), it has been found that a catheter having a circular cross-sectional shape in the radial direction is difficult to be curved, at a meandering portion in which three-dimensional meandering continues as in a coronary artery, along the shape of the meandering portion Further, as a result of studies by the present inventor(s), it has been found that, when the cross-sectional shape of a catheter in the radial direction is an elliptical shape, the catheter is easily curved along the shape of a meandering portion but is difficult to be pushed. As a result of further studies by the present inventor(s), it has been found that, when a second region in which a value obtained by dividing the major axis by the minor axis thereof in a cross-section in the radial direction is 1.2 to 2.0 is disposed, in a catheter, on the distal side of a first region in which a value obtained by dividing the major axis by the minor axis thereof is 1.0 to 1.1, the catheter is easily curved along the shape of a meandering portion and is easily pushed. Consequently, the catheter is easily inserted into a meandering portion in the body.

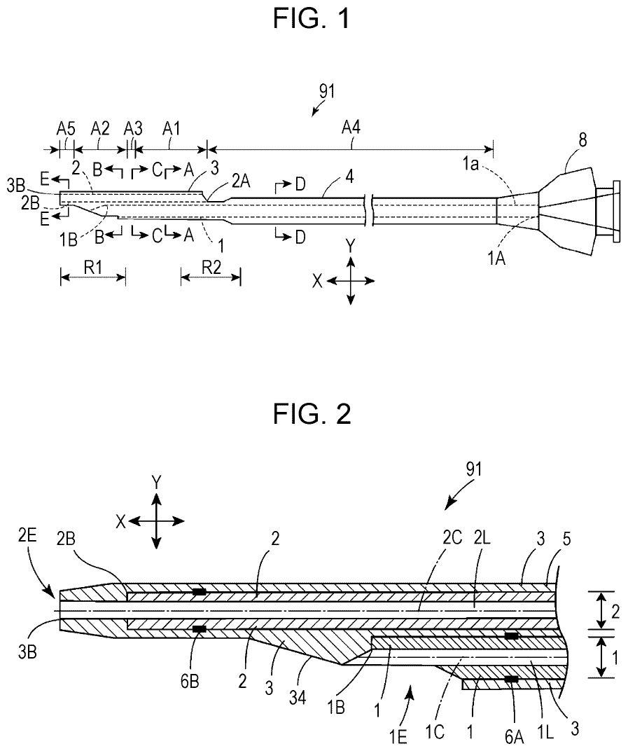

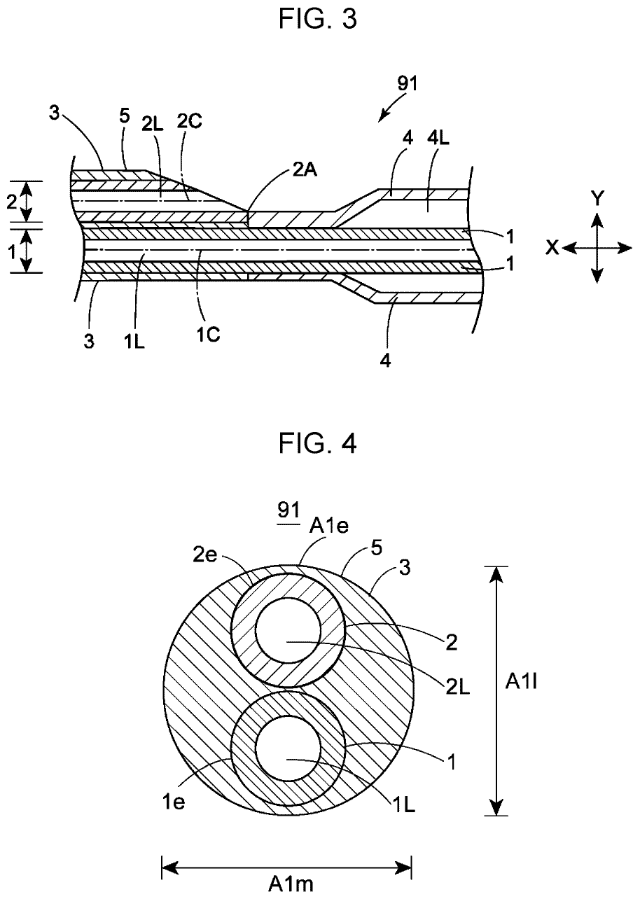

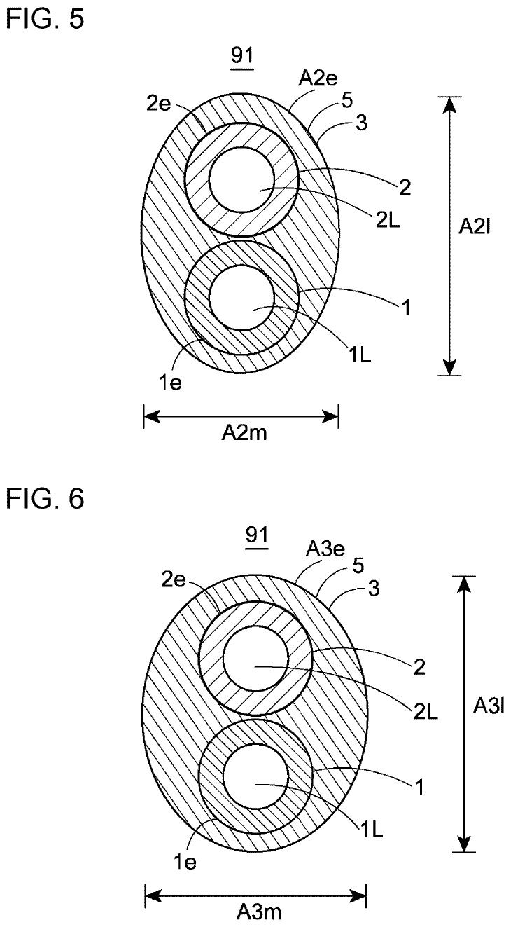

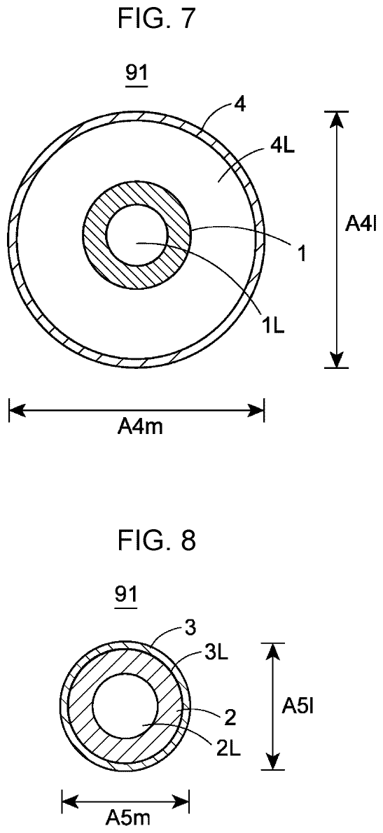

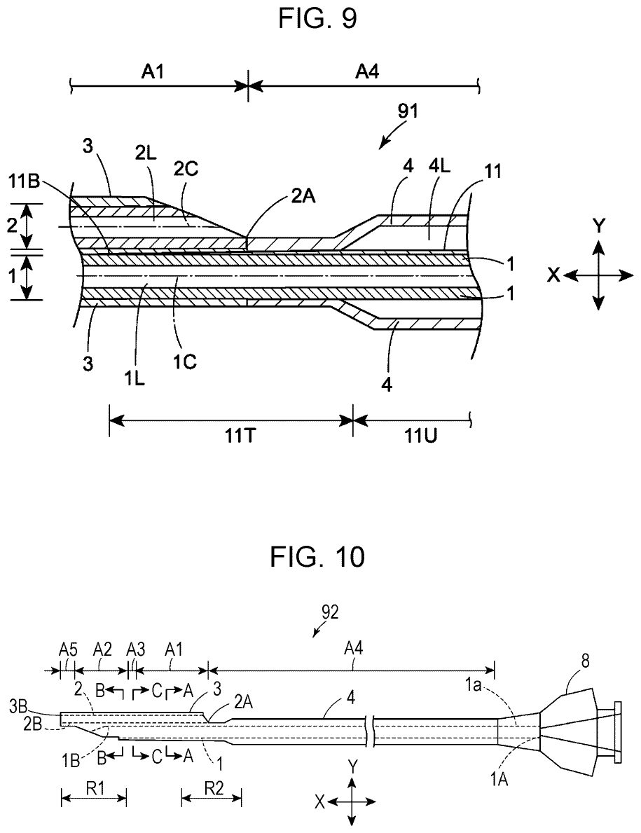

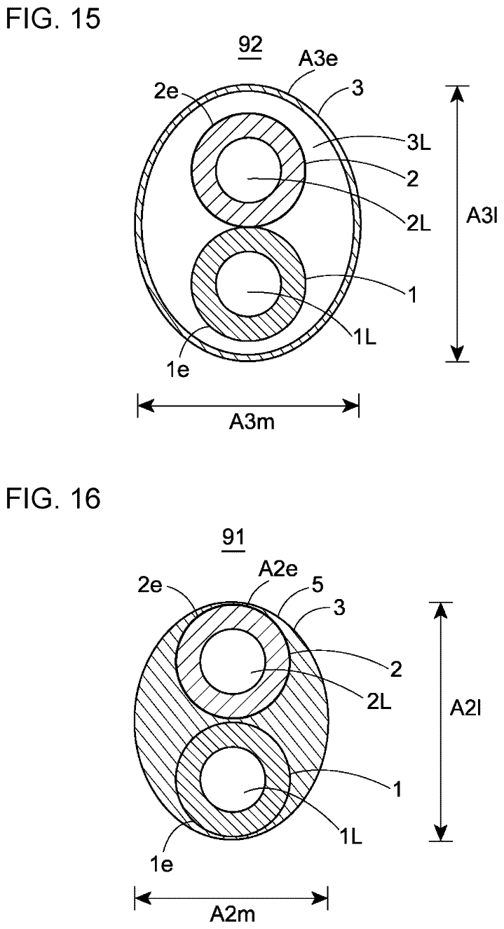

Hereinafter, a catheter according to a first embodiment will be described with reference to FIGS. 1 to 9 and 16. FIG. 1 is a side view of the catheter according to the first embodiment. FIG. 2 is a longitudinal sectional view of the region R1 in FIG. 1. FIG. 3 is a longitudinal sectional view of the region R2 in FIG. 1. FIG. 4 is a cross-sectional view of the catheter taken along line A-A in FIG. 1. FIG. 5 is a cross-sectional view of the catheter taken along line B-B in FIG. 1. FIG. 6 is a cross-sectional view of the catheter taken along line C-C in FIG. 1. FIG. 7 is a cross-sectional view of the catheter taken along line D-D in FIG. 1. FIG. 8 is a cross-sectional view of the catheter taken along line E-E in FIG. 1. FIG. 9 is a longitudinal sectional view of the region R2 in a modification of the catheter in FIG. 1. FIG. 16 is a cross-sectional view of a modification of the catheter in FIG. 5.

As illustrated in FIGS. 1, 2, and 3, the catheter according to the first embodiment is a catheter 91 including a first tubular member 1 extending in a longitudinal direction X, and a second tubular member 2 extending in the longitudinal direction X and adjacent to the first tubular member 1 in a radial direction Y. Specifically, it is preferable that an outer surface of the first tubular member 1 and an outer surface of the second tubular member 2 are adjacent to each other in the radial direction Y. The first tubular member 1 may have a lumen 1L extending in the longitudinal direction X. The second tubular member 2 may have a lumen 2L extending in the longitudinal direction X. For example, by inserting a guidewire into each of the lumen 1L and the lumen 2L, the catheter 91 can be guided in multiple directions. Therefore, the catheter 91 can be easily inserted into various branch portions of, for example, coronary arteries. In addition, by inserting a search wire into the lumen 1L or the lumen 2L, it is possible to easily search for an entrance or the like of a stenotic portion, which is a lesion site. It is preferable to insert the search wire into the lumen 1L. A distal end portion of the search wire may be bent.

Each of the first tubular member 1 and the second tubular member 2 may have one or a plurality of layers extending in the longitudinal direction X or may have a plurality of layers. The plurality of layers may include an inner layer and an outer layer that encloses at least a portion of the inner layer. The plurality of layers may further include an intermediate layer positioned between the inner layer and the outer layer. The number of intermediate layers is not limited to one and may be two or more. Each of the plurality of layers may contain a material different from a material of a layer adjacent thereto. The inner layer of the first tubular member 1 and the inner layer of the second tubular member 2 each may contain at least one resin selected from the group consisting of a polyolefin-based resin and a fluorine-based resin. Consequently, the slidability of a wire inserted into the lumen 1L and the lumen 2L can be improved. The polyolefin-based resin may be polyethylene, polypropylene, an ethylene-propylene copolymer, or a mixture thereof. The polyethylene may be high-density polyethylene having a specific gravity of 0.942 or greater. The fluorine-based resin may be polytetrafluoroethylene, a tetrafluoroethylene-perfluoroalkyl vinyl ether copolymer, a tetrafluoroethylene-hexafluoropropylene copolymer, a tetrafluoroethylene-ethylene copolymer, polyvinylidene fluoride, polychlorotrifluoroethylene, an ethylene-chlorotrifluoroethylene copolymer, or a mixture thereof. The inner layer of the first tubular member 1 and the inner layer of the second tubular member 2 may each contain at least one selected from the group consisting of a polyamide-based resin, a polyester-based resin, a polyurethane-based resin, a polyolefin-based resin, a fluorine-based resin, a vinyl chloride resin, a silicone-based resin, and natural rubber. The inner layer of the first tubular member 1 and the inner layer of the second tubular member 2 may contain resins different from each other, preferably contain the same resin and more preferably are made of the same resin.

Each of the outer layer of the first tubular member 1 and the outer layer of the second tubular member 2 may contain a flexible resin. When the outer layers each contain a flexible resin, the catheter 91 is easily curved. The flexible resin may contain at least one selected from the group consisting of polyethylene, polyurethane, a polyurethane-based thermoplastic elastomer, a styrene-based thermoplastic elastomer, a polyamide elastomer, and polyamide or may contain at least one selected from the group consisting of polyurethane and a polyamide elastomer. The polyethylene may be low-density polyethylene having a specific gravity of 0.91 to 0.92.

The Shore D hardness of the material of each outer layer may be less than or equal to, or may be less than, the Shore D hardness of the material of each inner layer. Consequently, the catheter 91 is easily curved. Shore D hardness may be measured, for example, with a Durometer Type D in accordance with ISO7619.

Each intermediate layer may contain a material different from the material of each inner layer or each outer layer, or may contain the same material as the material of each inner layer or each outer layer.

Each of the inner layer, the intermediate layer, and the outer layer may be fixed to a layer adjacent thereto. Consequently, each layer is less likely to be wrinkled or twisted. Examples of the fixing method include welding and adhesion. Although not illustrated, a reinforcing member may be disposed as the intermediate layer.

The first tubular member 1 may include a reinforcing member. Consequently, it is possible to easily prevent puncture of the first tubular member 1 by a wire. On the other hand, the second tubular member 2 may not be provided with a reinforcing member. Consequently, the flexibility of the second tubular member 2 can be easily improved.

The reinforcing member may be a braided body in which a wire material is braided, a coil body in which a wire material is spirally wound, or a combination thereof. Consequently, it is possible to easily prevent puncture of the first tubular member 1 by a wire. In particular, the braided body easily improves the rigidity of the reinforcing member. Thus, the reinforcing member may be the braided body. Examples of the wire material included in the reinforcing member include a metal wire and fiber. The metal wire may contain stainless steel, titanium, a nickel-titanium alloy, a cobalt-chromium alloy, a tungsten alloy, or alloys thereof. In particular, stainless steel is more preferable. The metal wire may be a single wire or a stranded wire. As the fiber, polyarylate fiber, aramid fiber, ultra-high-molecular-weight polyethylene fiber, PBO fiber, carbon fiber, or a mixed fiber thereof is preferable. The fiber may be monofilaments or multifilaments. The reinforcing member may contain a radiopaque substance so that the position of the first tubular member 1 can be easily confirmed under X-ray fluoroscopy or the like. The radiopaque substance may be at least one selected from the group consisting of lead, barium, iodine, tungsten, gold, platinum, iridium, a platinum-iridium alloy, stainless steel, titanium, a cobalt-chromium alloy, palladium, bismuth, and tantalum.

Although not illustrated, the distal end of the reinforcing member may be located on the proximal side of the proximal end of a first opening portion 1E and on the distal side of a proximal end 2A of the second tubular member 2. On the other hand, the proximal end of the reinforcing member may be located on the proximal side of the proximal end 2A of the second tubular member 2 or may be located in a handle portion 8. Consequently, it is possible to easily prevent puncture of the first tubular member 1 by a wire.

As illustrated in FIG. 2, a distal end 1B of the first tubular member 1 is located on the proximal side of a distal end 2B of the second tubular member 2. Consequently, the catheter 91 is easily curved at a portion on the distal side of the distal end 1B of the first tubular member 1.

As illustrated in FIGS. 1 and 3, a proximal end 1A of the first tubular member 1 is located on the proximal side of the proximal end 2A of the second tubular member 2. Consequently, a portion of the catheter 91 on the proximal side of the proximal end 2A of the second tubular member 2 can be formed to have, for example, a double-tube structure (coaxial structure) so as to have a structure different from the structure, in which the first tubular member 1 and the second tubular member 2 overlap each other in the radial direction Y, on the distal side of the proximal end 2A of the second tubular member 2 and so as to be exert a different function.

As illustrated in FIGS. 1, 2, and 3, the catheter 91 includes an outer member 3 extending in the longitudinal direction X and including a portion that encloses at least a portion of the first tubular member 1 and at least a portion of the second tubular member 2. The outer member 3 can protect the first tubular member 1 and the second tubular member 2 and can easily prevent puncture by a wire from inside the catheter 91.

The outer member 3 may contain at least one selected from the group consisting of polyethylene, polyurethane, a polyurethane-based thermoplastic elastomer, a styrene-based thermoplastic elastomer, a polyamide elastomer, and polyamide. In particular, at least one selected from the group consisting of polyurethane and a polyamide elastomer is preferable.

As illustrated in FIG. 1, the outer member 3 has a first region A1 distal to the proximal end 2A of the second tubular member 2 and extending in the longitudinal direction X and a second region A2 extending in the longitudinal direction X and located on the distal side of the first region A1. As illustrated in FIG. 4, the value of the ratio of a major axis A1l to a minor axis A1m of the first region A1 in a cross-section in the radial direction Y is 1.0 to 1.1. As illustrated in FIG. 5, the value of the ratio of a major axis A2l to a minor axis A2m of the second region A2 in a cross-section in the radial direction Y is 1.2 to 2.0. Since a value obtained by dividing the major axis A1l by the minor axis A1m of the first region A1 is 1.0 to 1.1, the catheter 91 is less likely to be twisted in a meandering portion in the body, and thus, a force from the proximal side toward the distal side is easily transmitted through the catheter 91. On the other hand, since a value obtained by dividing the major axis A2l by the minor axis A2m of the second region A2 on the distal side of the first region A1 is 1.2 to 2.0, the catheter 91 can be easily curved along the shape of a meandering portion in the body. The value obtained by dividing the major axis A2l by the minor axis A2m may be 1.2 to 1.9, 1.2 to 1.7, or 1.2 to 1.5. Note that it is sufficient for the outer member 3 to have the first region A1 located distal to the proximal end 2A of the second tubular member 2, and the first region A1 may be located on the distal side of the proximal end 2A of the second tubular member 2. The length of the major axis A1l of the first region A1 may be 0.5 mm or greater and 2.0 mm or less or 0.7 mm or greater and 1.6 mm or less.

In the present specification, the major axis of each region in a cross-section in the radial direction Y means the length of a longest line segment among line segments that each connect two points on the outer edge of the region in the cross-section in the radial direction Y and that each pass through the centroid of the cross-section. When the shape of the cross-section is the shape of a portion of an ellipse to be described later, the major axis of a circumscribed virtual ellipse is defined as the major axis of the cross-section in the radial direction Y. Further, in the present specification, the minor axis of each region in a cross-section in the radial direction Y means the length of a shortest line segment among line segments that each connect two points on the outer edge of the region in the cross-section in the radial direction Y and that each pass through the centroid of the cross-section. When the shape of the cross-section is the shape of a portion of an ellipse to be described later, the minor axis of a circumscribed virtual ellipse is defined as the minor axis in the cross-section in the radial direction Y. When the shape of the outer edge in the cross-section in the radial direction Y is circular, the length of the major axis and the length of the minor axis are the same.

As illustrated in FIG. 4, the shape of an outer edge A1e of the first region A1 in a cross-section in the radial direction Y may be a circle or an ellipse or may be a circle. Consequently, the first region A1 is unlikely to be twisted in a meandering portion in the body, and thus, a force from the proximal side toward the distal side is easily transmitted through the catheter 91. The shape of the outer edge A1e of the first region A1 in a cross-section in the radial direction Y may be a shape of, for example, a polygon with rounded corners. The first region A1 may include portions having cross-sectional shapes different from each other in the radial direction Y.

As illustrated in FIG. 5, the shape of an outer edge A2e of the second region A2 in a cross-section in the radial direction Y may be an ellipse. Consequently, the second region A2 is easily curved along the shape of a meandering portion in the body, and the catheter 91 is thus easily curved along the shape of the meandering portion in the body. In addition, for example, in the vicinity of the first opening portion 1E, the shape of the outer edge A2e of the second region A2 in a cross-section in the radial direction Y may be a shape of a portion of an ellipse. The shape of a portion of an ellipse is a shape of an ellipse from which a portion is removed. The shape of the outer edge A2e of the second region A2 in a cross-section in the radial direction Y may be a shape of, for example, a polygon with rounded corners. The second region A2 may include portions having cross-sectional shapes different from each other in the radial direction Y.

As illustrated in FIGS. 4 and 5, the area of the region surrounded by the outer edge A1e of the first region A1 in a cross-section in the radial direction Y may be larger than or equal to, or may be larger than, the area of the region surrounded by the outer edge A2e of the second region A2 in a cross-section in the radial direction Y. Consequently, the catheter 91 is more easily inserted into a meandering portion in the body. The area of the region surrounded by the outer edge A1e of the first region A1 in a cross-section in the radial direction Y may be not less than 1.00 times and not greater than 2.00 times or not less than 1.01 times and not greater than 1.50 times the area of the region surrounded by the outer edge A2e of the second region A2 in a cross-section in the radial direction Y.

The major axis A2l of the second region A2 may have a length longer than or equal to the length of the major axis A1l of the first region A1 or may have a length longer than the length of the major axis A1l of the first region A1. Consequently, a force from the proximal side toward the distal side is easily transmitted through the second region A2.

As illustrated in FIGS. 4 and 16, the major axis A2l of the second region A2 may be shorter than the major axis All of the first region A1. In this case, the flexibility of the second region A2 is improved.

As illustrated in FIGS. 4 and 5, in the radial direction Y, the shortest distance from the outer edge A2e of the second region A2 of the outer member 3 to an outer edge 1e of the first tubular member 1 may be longer than or equal to the shortest distance from the outer edge A1e of the first region A1 of the outer member 3 to the outer edge 1e of the first tubular member 1 or may be longer than the shortest distance from the outer edge A1e to the outer edge 1e. Consequently, for example, even when a wire is inserted into a meandering portion of the second region A2, the first tubular member 1 and the second region A2 are less likely to be punctured by the wire. The shortest distance of the second region A2 is measured in a portion of the second region A2 where the cross-sectional shape thereof in the radial direction Y is an ellipse. Hereinafter, the same applies to the shortest distance of the second region A2.

As illustrated in FIGS. 4 and 16, in the radial direction Y, the shortest distance from the outer edge A2e of the second region A2 of the outer member 3 to the outer edge 1e of the first tubular member 1 may be shorter than the shortest distance from the outer edge A1e of the first region A1 of the outer member 3 to the outer edge 1e of the first tubular member 1. In this case, the second region A2 in the vicinity of the first tubular member 1 is less likely to be wrinkled when being inserted into a meandering portion.

In the radial direction Y, the shortest distance from the outer edge A2e of the second region A2 of the outer member 3 to an outer edge 2e of the second tubular member 2 may be longer than or equal to the shortest distance from the outer edge A1e of the first region A1 of the outer member 3 to the outer edge 2e of the second tubular member 2 or may be longer than the shortest distance from the outer edge A1e to the outer edge 2e. Consequently, for example, even when a wire is inserted into a meandering portion of the second region A2, the second tubular member 2 and the second region A2 are less likely to be punctured by the wire. The shortest distance of the second region A2 is measured in a portion of the second region A2 where the cross-sectional shape thereof in the radial direction Y is an ellipse. Hereinafter, the same applies to the shortest distance of the second region A2.

As illustrated in FIGS. 4 and 16, in the radial direction Y, the shortest distance from the outer edge A2e of the second region A2 of the outer member 3 to the outer edge 2e of the second tubular member 2 may be shorter than the shortest distance from the outer edge A1e of the first region A1 of the outer member 3 to the outer edge 2e of the second tubular member 2. In this case, the second region A2 in the vicinity of the second tubular member 2 is less likely to be wrinkled when being inserted into a meandering portion.

As illustrated in FIG. 1, the length of the second region A2 in the longitudinal direction X may be shorter than the length of the first region A1 in the longitudinal direction X. Consequently, the catheter 91 is more easily inserted into a meandering portion in the body. To be specific, the length of the first region A1 in the longitudinal direction X may be not less than 1.1 times and not greater than 30 times or not less than 1.2 times and not greater 20 times the length of the second region A2 in the longitudinal direction X.

As illustrated in FIGS. 1 and 6, the outer member 3 has a third region A3 between the first region A1 and the second region A2 in the longitudinal direction X, and the value of the ratio of a major axis A3l to a minor axis A3m of the third region A3 in a cross-section in the radial direction Y may be greater than 1.1 and less than 1.2. The third region A3 makes it easy to avoid bending due to a sudden change in shape between the first region A1 and the second region A2. Therefore, it is more preferable that the first region A1 and the third region A3 are adjacent to each other in the longitudinal direction X and that the third region A3 and the second region A2 are adjacent to each other in the longitudinal direction X. The length of the third region A3 in the longitudinal direction X may be 1 mm or greater and 20 mm or less or 3 mm or greater and 10 mm or less.

In the third region A3, it is preferable that a value obtained by dividing the major axis A3l by the minor axis A3m decreases from the proximal side toward the distal side. Consequently, bending due to a sudden change in shape between the first region A1 and the second region A2 is more easily avoided. The shape of the outer edge A3e of the third region A3 in a cross-section in the radial direction Y may be an ellipse. Consequently, the above-described effect is easily exerted. The shape of the outer edge A3e of the third region A3 in a cross-section in the radial direction Y may be a shape of, for example, a polygon with rounded corners.

As illustrated in FIG. 2, a distal end 3B of the outer member 3 may be located on the distal side of the distal end 2B of the second tubular member 2. By thus disposing the outer member 3 formed of a flexible material, it is possible to improve the flexibility of the portion of the catheter 91 on the distal side of the distal end 2B of the second tubular member 2.

At least a portion of the inner surface of the outer member 3 may be fixed to outer surfaces of the first tubular member 1 and the second tubular member 2. Consequently, the outer member 3 is unlikely to be wrinkled or twisted. Examples of the fixing method include welding and adhesion.

The length from the distal end 3B of the outer member 3 to the distal end 2B of the second tubular member 2 in the longitudinal direction X may be greater than the maximum thickness of the second tubular member 2. Consequently, the flexibility in the vicinity of the distal end of the catheter 91 can be improved.

As illustrated in, for example, FIGS. 4, 5, and 6, the outer surfaces of the first tubular member 1 and the second tubular member 2 may be not in contact with each other or may be in contact with and fixed to each other. Examples of the fixing method include welding and adhesion. When the outer surface of the first tubular member 1 and the outer surface of the second tubular member 2 are not in contact with each other, the outer surfaces may be fixed to each other via the resin of the outer member 3. As illustrated in, for example, FIGS. 4, 5, and 6, in a cross-section of the catheter 91 in the radial direction Y, the shape of each of the first lumen 1L and the second lumen 2L may be a circular shape, an elliptical shape, an arcuate shape, or a combination thereof or may be a circular shape or an elliptical shape. Consequently, the catheter 91 is easily curved. In the catheter 91, it is preferable that the first lumen 1L and the second lumen 2L are not in communication with each other. In a cross-section in the radial direction Y, the area of the first lumen 1L may be larger than the area of the second lumen 2L, may be smaller than the area of the second lumen 2L, or may be the same as the area of the second lumen 2L.

As illustrated in FIG. 2, the first tubular member 1 may have the first opening portion 1E in communication with the lumen 1L. The first opening portion 1E can serve as an inlet or an outlet for a wire.

As illustrated in FIG. 2, the second tubular member 2 may have a second opening portion 2E in communication with the lumen 2L. The second opening portion 2E can serve as an inlet or an outlet for a wire.

The outer member 3 may include a raised portion 34, on the distal side of the distal end 1B of the first tubular member 1, at a portion near the first tubular member 1 in the radial direction Y, the raised portion 34 being raised in a direction away from a central axis 2C. Consequently, the distal end of a wire to be inserted into the first tubular member 1 can be guided in a direction different from that of the distal end of a wire to be inserted into the second tubular member 2. Therefore, it is possible to easily perform a treatment on, for example, a lesion in a bifurcated portion. In addition, it is thus possible to make the wires less likely to be entangled. The raised portion 34 may be adjacent to the distal end 1B of the first tubular member 1. Consequently, a wire that has emerged from the first opening portion 1E is easily guided by the raised portion 34.

As illustrated in FIG. 2, when a cross-section in the longitudinal direction X and including a central axis 1C of the first tubular member 1 and the central axis 2C of the second tubular member 2 is defined as a cross-section X, the raised portion 34 may include, in the cross-section X, a first inclined portion inclined in a direction away from the central axis 2C toward the distal side and a second inclined portion inclined so as to approach the central axis 2C toward the distal side. The second inclined portion may be located on the distal side of the first inclined portion. A wire that has emerged from the first opening portion 1E is easily guided by the first inclined portion. The second inclined portion facilitates insertion into the body. The shapes of outer surfaces of the first inclined portion and the second inclined portion in the cross-section X may be linear or curved or may be linear. When the outer surface of the second inclined portion in the cross-section X is linear, the shape of the second inclined portion is a so-Called tapered shape.

On the other hand, although not illustrated, the second tubular member 2 does not necessarily include the raised portion, which is raised in a direction away from the central axis 2C, at a portion near the first tubular member 1 in the radial direction Y on the distal side of the distal end 1B of the first tubular member 1. Consequently, the outer diameter of the catheter 91 is reduced by an amount corresponding to the raised portion, and the catheter 91 is thus easily curved.

The outer diameter of each of the first tubular member 1 and the second tubular member 2 may be 0.2 mm or greater and 0.8 mm or less or 0.3 mm or greater and 0.7 mm or less. Consequently, the catheter 91 is more easily inserted into a meandering portion of a coronary artery.

As illustrated in FIG. 1, the handle portion 8 may be disposed at a proximal end portion 1a of the first tubular member 1. To be specific, it is preferable that the proximal end portion 1a of the first tubular member 1 is disposed in the handle portion 8. An operator can insert the catheter 91 into the body by holding and pushing the handle portion 8. The outer shape of the handle portion 8 may be any shape as long as an operator can hold the handle portion 8 with fingers. The handle portion 8 may be a resin molded product obtained by injection molding or the like. Examples of the resin include polyolefin-based resins such as polyethylene and polypropylene, polycarbonate-based resins, and (meth) acrylic-based resins. Among these resins, transparent resins such as polycarbonate and polymethyl methacrylate are preferable. Consequently, the inside of the handle portion 8 can be easily seen, and a wire can be easily inserted.

The proximal end 1A of the first tubular member 1 may be located in the handle portion 8. Consequently, the pushability can be improved.

The handle portion 8 may have a lumen extending in the longitudinal direction X to be in communication with the lumen 1L of the first tubular member 1 so that a wire can be inserted through the lumen.

As illustrated in FIGS. 1 and 3, the catheter 91 may further include an outer tubular member 4 extending in the longitudinal direction X. The outer tubular member 4 may have a lumen 4L extending in the longitudinal direction X, and a portion of the first tubular member 1 may be disposed in the lumen 4L. A double-tube structure (coaxial structure) can be formed by the outer tubular member 4, and the transmissibility of a pushing force can be improved while the first tubular member 1 is protected. Specifically, it is preferable that the outer tubular member 4 includes, at a distal end portion, a fixed portion that is fixed to the first tubular member 1 and includes, on the proximal side of the fixed portion, a non-fixed portion that is not fixed to the first tubular member 1. With the non-fixed portion, it is possible to easily release a force accompanying the insertion of a wire into the first tubular member 1. Therefore, the non-fixed portion may be longer than the fixed portion in the longitudinal direction X. Consequently, it is possible to reduce the risk of puncture of the first tubular member 1 by a wire. In addition, the outer tubular member 4 may include, at a proximal end portion, a fixed portion fixed to the handle portion 8.

The outer tubular member 4 may be located on the proximal side of the proximal end 2A of the second tubular member 2. Consequently, the outer diameter of the outer tubular member 4 can be reduced.

The outer tubular member 4 may contain a resin that is the same as or harder than the resin of the outer member 3 or may be made of the harder resin. The harder resin can improve the pushability. In addition, the distal end of the outer tubular member 4 and the proximal end of the outer member 3 may be fixed to each other.

As illustrated in FIGS. 1 and 7, the outer tubular member 4 may have a fourth region A4 proximal to the proximal end 2A of the second tubular member 2 and extending in the longitudinal direction X, and the value of the ratio of a major axis A4l to a minor axis A4m of the fourth region A4 in a cross-section in the radial direction Y may be 1.0 to 1.1. When a value obtained by dividing the major axis A4l by the minor axis A4m is 1.0 to 1.1, the catheter 91 is less likely to be twisted in a meandering portion in the body, and thus, a force from the proximal side toward the distal side is easily transmitted therethrough.

It is sufficient for the outer tubular member 4 to have the fourth region A4 located proximal to the proximal end 2A of the second tubular member 2, and the fourth region A4 may be located on the proximal side of the proximal end 2A of the second tubular member 2.

As illustrated in FIGS. 5 and 7, it is preferable that the major axis A2l of the second region A2 is shorter than the major axis A4l of the fourth region A4. Consequently, the second region A2 is more easily curved at a meandering portion in the body. The major axis A2l may be not greater than 0.99 times or not greater than 0.98 times the major axis A4l. On the other hand, the major axis A2l may be not less than 0.5 times or not less than 0.6 times the major axis A4l. Consequently, the pushability of the second region A2 can be improved.

As illustrated in FIG. 1, the length of the fourth region A4 in the longitudinal direction X may be longer than the sum of the length of the first region A1 in the longitudinal direction X and the length of the second region A2 in the longitudinal direction X. Consequently, the pushability of the catheter 91 can be improved. In the longitudinal direction X, the length of the fourth region A4 may be not less than twice or not less than three times the sum of the lengths of the first region A1 and the second region A2. On the other hand, the multiplying factor may be not greater than 14 times or not greater than 6 times. Consequently, the length of the catheter 91 can be reduced. The sum of the lengths of the first region A1 and the second region A2 in the longitudinal direction X may be 5 cm or greater and 50 cm or less or 10 cm or greater and 40 cm or less.

As illustrated in FIGS. 1 and 8, the outer member 3 may have a fifth region A5 extending in the longitudinal direction X and located on the distal side of the second region A2, and the value of the ratio of a major axis A5l to a minor axis A5m of the fifth region A5 in a cross-section in the radial direction Y may be 1.0 to 1.1. When a value obtained by dividing the major axis A5l by the minor axis A5m is 1.0 to 1.1, it is easy to prevent deformation such as twisting in the vicinity of the distal end of the catheter 91. Therefore, the fifth region A5 may include the distal end of the catheter 91. The fifth region A5 may be adjacent to the second region A2 in the longitudinal direction X. However, another region may be present between the fifth region A5 and the second region A2. The value of the ratio of the major axis to the minor axis of the other region in a cross-section in the radial direction Y may be greater than 1.1 and less than 1.2. Consequently, bending due to a sudden change in shape is easily avoided.

As illustrated in, for example, FIGS. 4 to 6, the outer member 3 may include a columnar body 5 extending in the longitudinal direction X and containing a resin, and at least a portion of the first tubular member 1 and at least a portion of the second tubular member 2 may be disposed in the columnar body 5. That is, it is preferable that at least a portion of each of the first tubular member 1 and the second tubular member 2 is sealed by the columnar body 5 extending in the longitudinal direction X and containing a resin. Consequently, even when the catheter 91 is curved, the shapes of the lumen 1L and the lumen 2L extending in the longitudinal direction X are easily maintained. The columnar body 5 may have at least the first region A1 and the second region A2, and may further have the third region A3 and/or the fifth region A5. For the details of each region, the description of each region of the tubular outer member 3 described above may be referred to. The resin of the columnar body 5 may present between the first tubular member 1 and the second tubular member 2.

The columnar body 5 may include an elliptic columnar portion or a columnar portion or include an elliptic columnar portion and a columnar portion. It is more preferable that the columnar body 5 includes a plurality of elliptic columnar portions different from each other in value obtained by dividing the major axis by the minor axis thereof. The plurality of elliptic columnar portions may be adjacent to each other in the longitudinal direction X. Each elliptic columnar portion may be a portion in which a value obtained by dividing the major axis by the minor axis thereof is constant or may be a portion in which a value obtained by dividing the major axis by the minor axis thereof changes toward the distal side. When the columnar body 5 includes an elliptic columnar portion and a columnar portion, the elliptic columnar portion and the columnar portion may be adjacent to each other in the longitudinal direction X.

The columnar body 5 can be formed by, for example, disposing two or more tubes different from each other in value obtained by dividing the major axis by the minor axis thereof so as to be adjacent to each other in the longitudinal direction X, disposing the first tubular member 1 and the second tubular member 2 in a state of overlapping each other in the radial direction Y inside these tubes, and heating these tubes to be welded to the first tubular member 1 and the second tubular member 2. Alternatively, the columnar body 5 can be formed by preparing a tube in which a value obtained by dividing the major axis by the minor axis thereof is 1.0 to 1.1, disposing the first tubular member 1 and the second tubular member 2 in a state of overlapping each other in the radial direction Y inside the tube, and welding the tube to the first tubular member 1 and the second tubular member 2 while heating the tube and sandwiching a portion of the tube from two sides in the radial direction inwardly by plates or the like. In this case, the second region A2 can be formed by setting a value obtained by dividing the major axis by the minor axis of the portion sandwiched by the plates or the like to 1.2 to 2.0. The same applies to the other region.

As illustrated in FIG. 2, on the proximal side of the proximal end of the first opening portion 1E, the first tubular member 1 may include a radiopaque marker 6A on the distal side of a point spaced apart by 1 cm toward the proximal side from the proximal end of the first opening portion 1E. Consequently, the position of the first opening portion 1E can be easily grasped.

The second tubular member 2 may include, on the distal side of the distal end 1B of the first tubular member 1, a radiopaque marker 6B at a position on the proximal side of the distal end 2B of the second tubular member 2. Consequently, when the catheter 91 is inserted into a meandering portion in the body, the position of a curved portion of the catheter 91 can be easily grasped, and thus, the insertion can be easily performed.

The radiopaque marker may have a ring shape. The radiopaque marker may contain a radiopaque substance or may be made of a radiopaque substance. The radiopaque substance may be at least one selected from the group consisting of lead, barium, iodine, tungsten, gold, platinum, iridium, a platinum-iridium alloy, stainless steel, titanium, a cobalt-chromium alloy, palladium, bismuth, and tantalum.

As illustrated in FIG. 9, the catheter 91 may include a wire member 11 extending in the longitudinal direction X to be disposed in at least the first region A1 and the fourth region A4 and having a distal end 11B located in the first region A1. Thus, the pushability can be improved.

The wire member 11 may contain a metal or may be made of a metal. The metal may be stainless steel, nickel titanium, cobalt chromium, or an alloy thereof. Consequently, the pushability can be further improved.

The shape of the wire member 11 may be linear. The wire member 11 may be a solid member having no lumen. Consequently, the pushability can be further improved. The number of the wire members 11 included in the catheter 91 may be one or may be two or more.

The wire member 11 may include a constant-diameter portion 11U extending in the longitudinal direction X and having a constant length in the radial dimension. It is more preferable that the wire member 11 further includes a tapered portion 11T located on the distal side of the constant-diameter portion 11U and tapered toward the distal side.

Although not illustrated, the proximal end of the wire member 11 may be located in the handle portion 8. A proximal end portion of the wire member 11 may be fixed to the first tubular member 1, the handle portion 8, or the outer tubular member 4. Consequently, the pushability can be further improved.

Although not illustrated, the catheter 91 may further include a tip extending in the longitudinal direction X. The tip may have a lumen extending in the longitudinal direction X, and at least a distal end portion of the second tubular member 2 may be disposed in the lumen. Consequently, the tip and the second tubular member 2 are easily curved integrally.

At least a portion of an inner surface of the tip may be fixed to the outer surface of the second tubular member 2. Consequently, the tip and the second tubular member 2 are more easily curved integrally. Examples of the fixing method include welding and adhesion.

The chip may contain a flexible resin. The flexible resin may contain at least one selected from the group consisting of polyethylene, polyurethane, a polyurethane-based thermoplastic elastomer, a styrene-based thermoplastic elastomer, a polyamide elastomer, and polyamide or contain at least one selected from the group consisting of polyurethane and a polyamide elastomer. The polyethylene may be low-density polyethylene having a specific gravity of 0.91 to 0.92.

The tip may have a region in which the value of the ratio of the major axis to the minor axis thereof in a cross-section in the radial direction Y is 1.0 to 1.1. The region may include the distal end of the tip. Consequently, deformation such as twisting of the distal end portion of the tip is easily prevented. The chip may be constituted by the region.

The catheter 91 may be used for a treatment for expanding a stenotic or occluded site of a passage such as a blood vessel in the body or may be used by performing percutaneous coronary intervention (PCI) on, for example, a lesion in a bifurcated portion of a coronary artery.

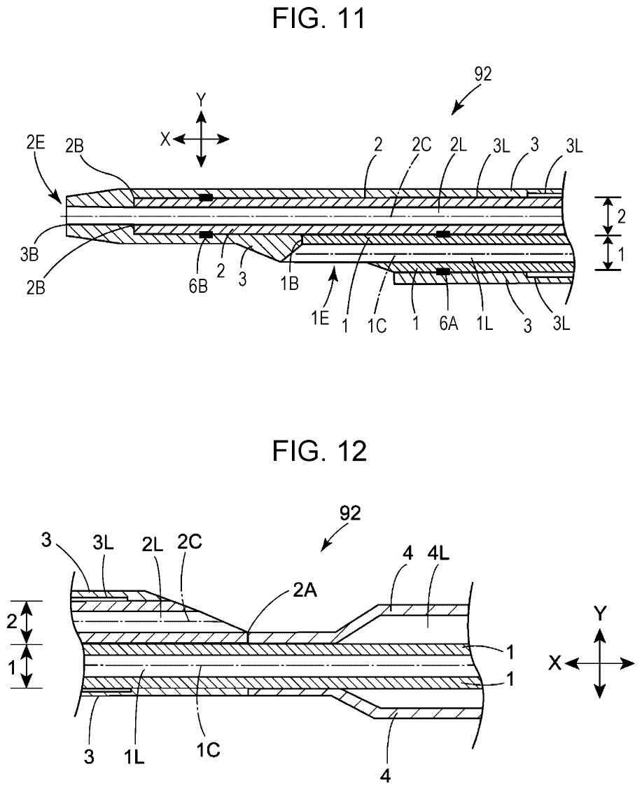

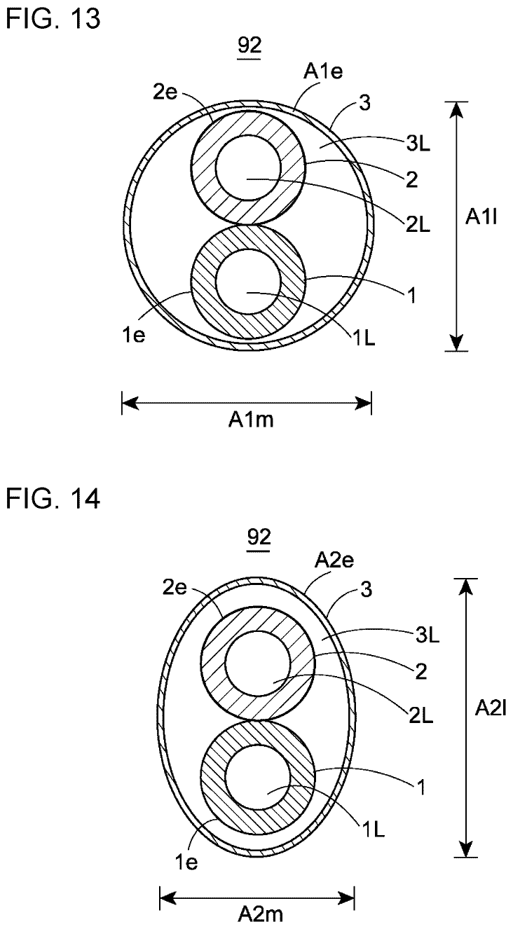

Hereinafter, a catheter according to a second embodiment will be described with reference to FIGS. 10 to 15. FIG. 10 is a side view of the catheter according to the second embodiment. FIG. 11 is a longitudinal sectional view of the region R1 in FIG. 10. FIG. 12 is a longitudinal sectional view of the region R2 in FIG. 10. FIG. 13 is a cross-sectional view of the catheter taken along line A-A in FIG. 10. FIG. 14 is a cross-sectional view of the catheter taken along line B-B in FIG. 10. FIG. 15 is a cross-sectional view of the catheter taken along line C-C in FIG. 10. Detailed description of, in the catheter according to the second embodiment, portions common to the catheter according to the first embodiment will be omitted.

The catheter according to the second embodiment is a catheter 92 including the first tubular member 1 extending in the longitudinal direction X, the second tubular member 2 extending in the longitudinal direction X and adjacent to the first tubular member 1 in the radial direction Y, and the outer member 3 extending in the longitudinal direction X and including a portion that encloses at least a portion of the first tubular member 1 and at least a portion of the second tubular member 2. For details of each member, the description of the catheter 91 may be referred to.

As illustrated in FIGS. 10 to 15, in the catheter 92, the outer member 3 may have a tubular shape and have a lumen 3L extending in the longitudinal direction X, and at least a portion of the first tubular member 1 and at least a portion of the second tubular member 2 may be disposed in the lumen 3L. Consequently, the first tubular member 1 and the second tubular member 2 can be protected, and each tubular member can move to some extent due to a gap between the outer member 3 and the first tubular member 1 and a gap between the outer member 3 and the second tubular member 2. Therefore, the force of a wire from inside the catheter 92 can be released. As a result, puncture by the wire can be easily prevented. A distal end portion of the tubular outer member 3 may be fixed to the first tubular member 1 and the second tubular member 2. A proximal end portion of the tubular outer member 3 may be fixed to the first tubular member 1 and the second tubular member 2. Consequently, the tubular outer member 3 is less likely to be wrinkled or twisted. Examples of the fixing method include welding and adhesion.

The first region A1, the second region A2, and the like of the tubular outer member 3 can be formed, for example, by joining two or more tubes, which are different from each other in value obtained by dividing the major axis by the minor axis thereof, in the longitudinal direction X. Alternatively, the second region A2 in which a value obtained by dividing the major axis by the minor axis thereof is 1.2 to 2.0 may be formed by preparing a tube in which a value obtained by dividing the major axis by the minor axis thereof is 1.0 to 1.1 and sandwiching the tube from two sides in the radial direction inwardly by plates or the like while heating a portion of the tube. The same applies to the other region.

The tubular outer member 3 has at least the first region A1 and the second region A2 and may further have the third region A3 and/or the fifth region A5. For the details of each region, the description of each region of the tubular outer member 3 described above may be referred to.

In the catheter 92, it is preferable that the first tubular member 1 and the second tubular member 2 are fixed to each other in the radial direction Y. Specifically, it is preferable that the outer surface of the first tubular member 1 and the outer surface of the second tubular member 2 are fixed to each other in the radial direction Y. Consequently, the first tubular member 1 and the second tubular member 2 are less likely to be wrinkled or twisted. Examples of the fixing method include welding and adhesion.

The present application claims the benefit of priority based on Japanese Patent Application No. 2023-125557 filed on Aug. 1, 2023. The entire content of the description in Japanese Patent Application No. 2023-125557 filed on Aug. 1, 2023 is incorporated herein by reference.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

### REFERENCE SIGNS LIST

1 first tubular member
1a proximal end portion
1e outer edge
1A proximal end
1B distal end
1C central axis
1E first opening portion
1L lumen
2 second tubular member
2e outer edge
2A proximal end
2B distal end
2C central axis
2E second opening portion
2L lumen
3 outer member
3B distal end
3L lumen
4 outer tubular member
4L lumen
5 columnar body
6A, 6B radiopaque marker
8 handle portion
11 wire member
11B distal end
11U constant-diameter portion
11T tapered portion
34 raised portion
91, 92 catheter
A1 first region
A1e outer edge
A1l major axis
A1m minor axis
A2 second region
A2e outer edge
A2l major axis
A2m minor axis
A3 third region
A3e outer edge
A3l major axis
A3m minor axis
A4 fourth region
A4l major axis
A4m minor axis
A5 fifth region
A5l major axis
A5m minor axis
X longitudinal direction
Y radial direction

1. A catheter comprising:
a first tubular member extending in a longitudinal direction;
a second tubular member extending in the longitudinal direction and adjacent to the first tubular member in a radial direction; and
an outer member extending in the longitudinal direction and including a portion that encloses at least a portion of the first tubular member and at least a portion of the second tubular member,
wherein a distal end of the first tubular member is located on a proximal side of a distal end of the second tubular member,
wherein a proximal end of the first tubular member is located on a proximal side of a proximal end of the second tubular member, and
wherein the outer member has a first region distal to the proximal end of the second tubular member and extending in the longitudinal direction and a second region extending in the longitudinal direction and located on a distal side of the first region,
wherein a ratio of a major axis to a minor axis of the first region in a cross-section in the radial direction is 1.0 to 1.1, and a ratio of a major axis to a minor axis of the second region in a cross-section in the radial direction is 1.2 to 2.0.

2. The catheter according to claim 1,
wherein a shape of an outer edge of the first region in a cross-section in the radial direction is a circle or an ellipse, and
wherein a shape of an outer edge of the second region in a cross-section in the radial direction is an ellipse or a shape of a portion of an ellipse.

3. The catheter according to claim 1, wherein the outer member has a third region between the first region and the second region in the longitudinal direction, and a ratio of a major axis to a minor axis of the third region in a cross-section in the radial direction is greater than 1.1 and less than 1.2.

4. The catheter according to claim 1, wherein a length of the second region in the longitudinal direction is shorter than a length of the first region in the longitudinal direction.

5. The catheter according to claim 1, wherein an area of a region surrounded by an outer edge of the first region in a cross-section in the radial direction is larger than or equal to an area of a region surrounded by an outer edge of the second region in a cross-section in the radial direction.

6. The catheter according to claim 1, wherein the major axis of the second region is greater than or equal to the major axis of the first region.

7. The catheter according to claim 1, wherein the major axis of the second region is shorter than the major axis of the first region.

8. The catheter according to claim 1, wherein, in the radial direction, a shortest distance from an outer edge of the second region of the outer member to an outer edge of the first tubular member is longer than or equal to a shortest distance from an outer edge of the first region of the outer member to the outer edge of the first tubular member.

9. The catheter according to claim 1, wherein, in the radial direction, a shortest distance from an outer edge of the second region of the outer member to an outer edge of the first tubular member is shorter than a shortest distance from an outer edge of the first region of the outer member to the outer edge of the first tubular member.

10. The catheter according to claim 1, wherein, in the radial direction, a shortest distance from an outer edge of the second region of the outer member to an outer edge of the second tubular member is longer than or equal to a shortest distance from an outer edge of the first region of the outer member to the outer edge of the second tubular member.

11. The catheter according to claim 1, wherein, in the radial direction, a shortest distance from an outer edge of the second region of the outer member to an outer edge of the second tubular member is shorter than a shortest distance from an outer edge of the first region of the outer member to the outer edge of the second tubular member.

12. The catheter according to claim 1, wherein the outer member is tubular and has a lumen extending in the longitudinal direction, and at least a portion of the first tubular member and at least a portion of the second tubular member are disposed in the lumen.

13. The catheter according to claim 1,
wherein the outer member includes a columnar body extending in the longitudinal direction and containing a resin, and
wherein at least a portion of the first tubular member and at least a portion of the second tubular member are disposed in the columnar body.

14. The catheter according to claim 1, further comprising:
an outer tubular member extending in the longitudinal direction,
wherein the outer tubular member has a lumen extending in the longitudinal direction, and a portion of the first tubular member is disposed in the lumen.

15. The catheter according to claim 14, wherein the outer tubular member is located on a proximal side of the proximal end of the second tubular member.

16. The catheter according to claim 14, wherein the outer tubular member has a fourth region proximal to the proximal end of the second tubular member and extending in the longitudinal direction, wherein a ratio of a major axis to a minor axis of the fourth region in a cross-section in the radial direction is 1.0 to 1.1.

17. The catheter according to claim 16, further comprising a wire member extending in the longitudinal direction and disposed in at least the first region and the fourth region, wherein the wire member has a distal end located in the first region.

18. The catheter according to claim 1, wherein the outer member has a fifth region extending in the longitudinal direction and located on a distal side of the second region, and a ratio of a major axis to a minor axis of the fifth region in a cross-section in the radial direction is 1.0 to 1.1.

19. The catheter according to claim 1, wherein the first tubular member and the second tubular member are fixed to each other in the radial direction.

20. The catheter according to claim 1, wherein a handle portion is disposed at a proximal end portion of the first tubular member.
